(19) United States
(12) Patent Application Publication
(10) Pub. No.: US 2004/0115779 A1
(43) Pub. Date: Jun. 17, 2004

(54) Fermentation process

(76) Inventors: Hans Sejr Olsen, Holte (DK); Sven Pedersen, Gentofte (DK); Robert Beckerich, Wendell, NC (US); Christopher Veit, Wake Forest, NC (US); Claus Felby, Veksoe (DK)

Correspondence Address: NOVOZYMES NORTH AMERICA, INC., 500 FIFTH AVENUE, SUITE 1600, NEW YORK, NY, 10110, US

(21) Appl. No.: 10/472,256

(22) PCT Filed: Mar. 19, 2002
(86) PCT No.: PCT/DK02/00179

(51) Int. Cl. C12P 19/02; C12N 1/16

(52) U.S. Cl. 435/105; 435/254.3; 435/252.31

(57) ABSTRACT

The present invention relates to an improved process for producing a fermentation product.

### FIELD OF THE INVENTION

The present invention relates to an improved fermentation process. More specifically the invention relates to a process for producing a fermentation product, in particular ethanol; a composition comprising at least a carbohydrate-source generating enzyme activity and at least an alpha-amylase activity and/or one or more yeast cell wall degrading enzymes, such as an enzyme preparation from Trichoderma, in particular T. harzianum sold under the tradename GLUCANEX™; and the use of the composition of the invention for saccharification and/or fermentation product, in particular ethanol production.

### BACKGROUND OF THE INVENTION

Fermentation processes are used for making a vast number of products of big commercial interest. Fermentation is used in industry to produce simple compounds such as alcohols (in particular ethanol); acids, such as citric acid, itaconic acid, lactic acid, gluconic acid; ketones; amino acids, such as glutamic acid, but also more complex compounds such as antibiotics, such as penicillin, tetracyclin; enzymes; vitamins, such as riboflavin, B₁₂, beta-carotene; hormones, which are difficult to produce synthetically. Also in the brewing (beer and wine industry), dairy, leather, tobacco industries fermentation processes are used.

There is a need for further improvement of fermentation processes and for improved processes including a fermentation step. Accordingly, the object of the invention is to provide an improved method at least comprising a fermenting step.

### BRIEF DESCRIPTION OF THE DRAWING

FIG. 1 shows schematically an ethanol production process of the invention.

FIG. 2 shows the indexed CO₂ loss in the fermentation step of an ethanol process of the invention without backset of mash.

FIG. 3 shows the indexed CO₂ loss in the fermentation step of an ethanol process of the invention with backset of mash.

FIG. 4 shows the fermentation yield based on 69% starch content.

### DETAILED DESCRIPTION OF THE INVENTION

The present invention relates to an improved process of producing a fermentation product, in particular ethanol, but also for instance the products mentioned in the “Background of the Invention”-section. Also beverage production, such as beer or wine production is contemplated according to the invention.

In the first aspect the invention relates to a process for producing a fermentation product, comprising a fermentation step, wherein the fermentation step is carried out in the presence of all of the following enzyme activities: carbohydrate-source generating enzyme activity, alpha-amylase activity, protease activity and debranching enzyme activity and/or one or more yeast cell wall degrading enzymes.

It is to be understood that the enzyme activities may according to the invention be added during pre-saccharification, the propagation of fermenting organism cells and/or later on during the actual fermentation.

Performing the fermentation step in the presence of all the activities of the invention have been shown to result in increased fermentation rate and ethanol yield.

Performing the fermentation step in the presence of a yeast cell wall degrading enzyme is advantageous at least partly due to the fact that the fermenting organism cells are hydrolysed during fermentation. This in situ hydrolysis of dead cells generates essential nutrients to the fermenting organism(s). Another advantage is that the yeast cell wall degrading enzyme(s) improve the yeast viability as it maintains a “fresh” population of fermenting cells throughout the fermentation. Further, fermenting in the presence of at least one yeast cell wall degrading enzyme result in increased fermentation rate and ethanol yield.

From Example 3 it can be seen that the yeast cell wall degrading preparation (i.e., GLUCANEX™) has a high effect on the initial fermentation rate: After only 24 hours using AMG E and GLUCANEX™ the ethanol yield was 96-97% as compared to using AMG E only. Furthermore, the ethanol yield was increased approximately 10%. The higher ethanol yields indicate that the corn cell wall glucans are hydrolyzed/converted to glucose.

The yeast cell wall degrading enzyme(s) may be selected from the group including beta-1,3-glucanase, 1,3-beta-glucanase, laminarinase, xylanase, chitinase, mannanase, alpha-1,3-glucanase (mutanase). In a preferred embodiment the yeast cell wall degrading enzyme is a preparation derived from Trichoderma, in particular Trichoderma harzianum, such as the product GLUCANEX™ (available from Novozymes A/S) derived from Trichoderma harzianum.

In preferred embodiment the addition of one or more yeast cell wall degrading enzymes is made in combination with at least one “carbohydrate-source generating enzyme” or alternatively in the presence of a for the fermenting organism suitable carbohydrate source. The term “carbohydrate-source generating enzyme” includes glucoamylases (being a glucose generator), and beta-amylases and maltogenic amylases (being maltose generators). The carbohydrate-source generating enzymes are this way capable of providing energy to the fermenting microorganism(s) in question and/or may be converting directly or indirectly to the desired fermentation product.

Further, it is to be understood that the enzymes used according to the invention should be added in effective amounts.

Carbohydrate-Source Generating Enzyme

The term “carbohydrate-source generating enzyme” includes glucoamylases (being a glucose generator), and beta-amylases and maltogenic amylases (being maltose generators). The carbohydrate-source generating enzymes are this way capable of providing energy to the fermenting microorganism(s) In question and/or may be converting directly or indirectly to the desired fermentation product.

It is to be understood that the enzymes used according to the invention should be added in effective amounts.

Debranching Enzymes

Debranching enzyme, include according to the present invention isoamylases and pullulanases. Debranching enzymes, which can attack amylopectin are divided into two classes: isoamylases (E.C. 3.2.1.68) and pullulanases (E.C. 3.2.1.41), respectively. Isoamylase hydrolyses alpha-1,6-D-glucosidic branch linkages in amylopectin and beta-limit dextrins and can be distinguished from pullulanases by the inability of isoamylase to attack pullulan, and by the limited action on alpha-limit dextrins.

It is to be understood that the enzymes used according to the invention should be added in effective amounts.

Ethanol Production

The process of the invention may in one embodiment be an ethanol process comprising the below steps, wherein the enzymatic activities are added during pre-saccharification and/or during fermentation. It is to be understood that the cell wall degrading enzyme(s) may according, to the invention be added during the propagation of yeast cells and/or later on during the actual fermentation. Beverage production, such as beer or wine production is equally contemplated.

Alcohol production, in particular ethanol production, from whole grain can be separated into 4 main steps

Milling

Liquefaction

Saccharification

Fermentation

Distillation

The individual process steps of alcohol production may be performed batch wise or as a continuous flow. For the invention processes where one or more process step(s) is(are) performed batch wise or one or more process step(s) is(are) performed as a continuous flow, are equally contemplated. Thus contemplated are also processes where the fermentation step is performed as a continuous flow. The cascade process is an example of a process where one or more process step(s) is(are) performed as a continuous flow and as such contemplated for the invention. Further information on the cascade process and other ethanol processes can be found in, e.g., “The Alcohol Textbook” Eds. T. P. Lyons, D. R. Kesall and J. E. Murtagh. Nottingham University Press 1995.

Milling

The raw material, such as whole grain, is milled in order to open up the structure and allowing for further processing. Two processes are preferred according to the invention: wet and dry milling. Preferred for ethanol production is dry milling where the whole kernel is milled and used in the remaining part of the process. Wet milling is Wet milling may also be used and gives a good separation of germ and meal (starch granules and protein) and is with a few exceptions applied at locations where there is a parallel production of syrups. Both dry and wet milling is well known in the art of, e.g., ethanol production.

Liquefaction

In an embodiment of the liquefaction step of the invention, milled gelatinized (whole) grain raw material is broken down (hydrolyzed) into maltodextrins (dextrins) mostly of a DE higher than 4. The hydrolysis may be carried out by acid treatment or enzymatically by alpha-amylase treatment, in particular with Bacillus alpha-amylases as will be described further below. Acid hydrolysis is used on a limited basis. The raw material is in one embodiment of the invention milled whole grain. However, a side stream from starch processing may also be used.

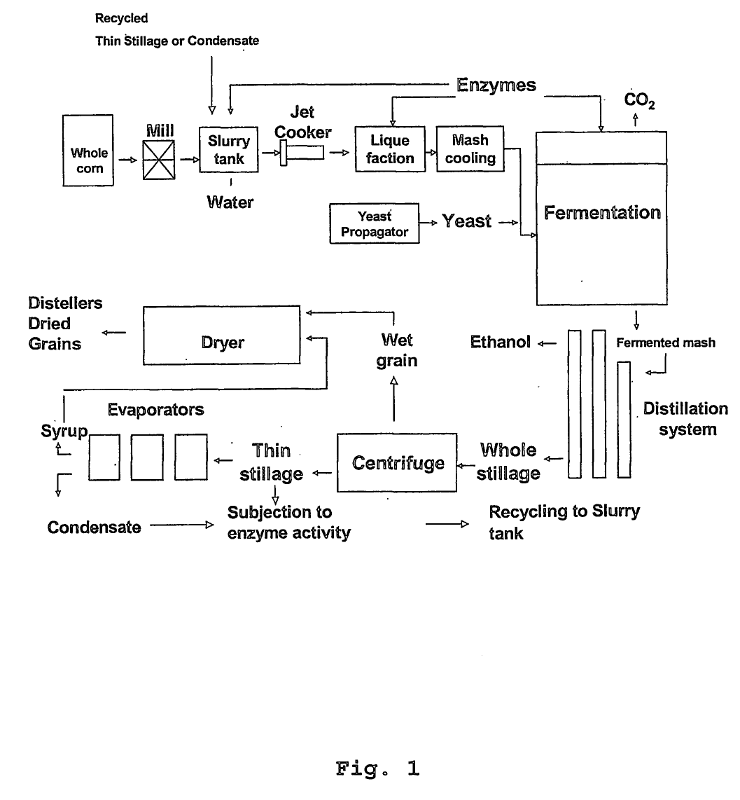

In an embodiment of the invention enzymatic liquefaction is carried out as a three-step hot slurry process. The slurry is heated to between 60-95° C., preferably 80-85° C. (in the Slurry Tank—see FIG. 1), and the enzyme(s) is(are) added to initiate liquefaction (thinning). Then the slurry is jet-cooked at a temperature between 95-140° C., preferably 105-125° C. to complete gelanitization of the slurry. Then the slurry is cooled to 60-95° C. and more enzyme(s) is(are) added to finalize hydrolysis (secondary liquefaction). The liquefaction process is carried out at pH 4.5-6.5, in particular at a pH between 5 and 6. Milled and liquefied whole grains are known as mash.

Saccharification

To produce low molecular sugars DP₁₋₃ (i.e., carbohydrate source) that can be metabolized by the fermenting organism, in particular yeast, the maltodextrin from the liquefaction must be further hydrolyzed. The hydrolysis may be done enzymatically and a typically done using a glucoamylase: Alternatively alpha-glucosidases or acid alpha-amylases may be used. As also mentioned above the carbohydrate source may be supplied by direct addition of, e.g., glucose or maltose.

A full saccharification step may last up to from 24 to 72 hours, however, it is common only to do a pre-saccharification of typically 40-90 minutes at 30-65° C., typically at about 60° C. and then complete saccharification during fermentation (SSF). Saccharification is typically carried out at temperatures from 30-65° C., typically around 60° C., and at a pH between 4 and 5, normally at about pH 4.5.

Fermentation

Which fermenting organism is suitable depends on the desired fermentation product. In the case of alcohol production, in particular ethanol production, the fermenting organism may be yeast, in particular derived from Saccharomyces spp., especially Saccharomyces cerevisiae, which is added to the mash and the fermentation is ongoing for 24-96 hours, such as typically 35-60 hours. The temperature is between 26-34° C., in particular about 32° C., and the pH is from pH 3-6, preferably around pH 4-5.

Note that the most widely used process is a simultaneous saccharification and fermentation (SSF) process where there is no holding stage for the saccharification, meaning that fermenting organism, such as the yeast, and enzyme(s) is(are) added together. When doing SSF it is common to introduce a pre-saccharification step at a temperature above 50° C., just prior to the fermentation. Among the variations of the SSF process especially the SSYPF (simultaneous saccharification, yeast propagation and fermentation) process has been applied. For the invention SSF and SSYPF processes are equally contemplated. Further information on SSF and SSYPF processes may be found in, e.g., “The Alcohol Textbook” Editors. T. P. Lyons, D. R. Kesall and J. E. Murtagh. Nottingham University Press 1995.

Distillation

Optionally following the fermentation, the mash may be distilled to extract the, e.g., alcohol product, in particular ethanol.

In the case where the end product is ethanol, obtained according to the process of the invention, it may be used as, e.g., fuel ethanol; drinking ethanol, i.e., potable neutral spirits or industrial ethanol.

By-Products

Left over from the fermentation or distillation steps is the grain, which is typically used for animal feed either in liquid or dried form.

### EMBODIMENTS OF THE INVENTION

Further details on how to carry out liquefaction, saccharification, fermentation, distillation and recovering of ethanol are well known to the skilled person.

According to the process of the invention the saccharification and fermentation may be carried out simultaneously or separately.

In one embodiment the invention relates to a process for the production of ethanol, comprising the steps of:

(a) milling whole grains; (b) liquefying the product of step (a) in the presence of an alpha-amylase, (c) saccharifying the liquefied material obtained in step (b) in the presence of a phytase, (d) fermenting the saccharified material obtained in step (c) using a micro-organism; and optionally (e) distilling of the fermented and saccharified material obtained in step (d), providing two fraction: 1) an alcohol fraction and 2) a Whole Stillage fraction; (f) separating the Whole Stillage into two fractions: 1) Wet Grain fraction, and 2) Thin Stillage; (g) optionally the Thin Stillage is evaporated to provide two fractions: 1) Condensate and 2) Syrup, wherein the steps (c) and/or (d) are carried out In the presence of at least one carbohydrate-source generating enzyme activity and at least one alpha-amylase activity and/or one or more yeast cell wall degrading enzyme, in particular one or more activities as defined as cell wall degrading herein. In one embodiment protease activity and/or a debranching enzyme activity is(are) present as well. Preferred examples of enzymes are described below in the “ENZYMES” section.

In a preferred embodiment the whole grains in step a) are dry milled, for instance in a hammer mill.

In an embodiment the DS % (dry solid percentage) in the slurry tank (containing milled whole grains) is in the range from 1-60%, in particular 10-50%, such as 20-40%, such as 25-35%.

In a preferred embodiment of the invention the liquefaction step comprising the following sub-steps:

b1) the hot slurry is heated to between 60-95° C., preferably 80-85° C., and at least an alpha-amylase is added;

b2) the slurry is jet-cooked at a temperature between 95-140° C., preferably 105-125° C. to complete gelanitization of the slurry;

b3) the slurry is cooled to 60-95° C. and more alpha-amylase is added to finalize hydrolysis.

The liquefaction process is in an embodiment carried out at pH 4.5-6.5, in particular at a pH between 5 and 6.

Steps (c) and (d) may be carried out either simultaneously or separately/sequential. Further, after step (e) an optional ethanol recovery step may be added.

Materials Used in Processes of the Invention

Raw material

Raw materials may be any starch-containing raw materials, such as tubers, roots, whole grains, corns, cobs, wheat, barley, rye, milo or cereals, sugar-containing raw materials, such as molasses, fruit materials, sugar, cane or sugar beet, potatoes, cellulose-containing materials, such as wood or plant residues.

According to the invention the raw material may be the side stream from starch processing, in particular liquefied starch with a DE of 6-20, in particular between 8-10.

Microorganism for Fermentation

Suitable micro-organisms used for fermentation according to the invention are capable of fermenting sugars or converted sugars, such as glucose or maltose, directly or indirectly into the desired fermentation product. Examples of contemplated microorganisms include fungal organisms, such as yeast. Examples of specific filamentous fungi include strains of Penicillium sp. Preferred organisms for ethanol production is yeast. Preferred yeast according to the invention is baker's yeast, also known as Saccharomyces cerevisiae. The yeast may according to the invention preferably be added before starting the actual fermentation (i.e., during the propagation phase). The yeast cells may be added in amounts of 10⁵ to 10¹², preferably from 10⁷ to 10¹⁰, especially 5×10⁷ viable yeast count per ml of fermentation broth. During the ethanol producing phase the yeast cell count should preferably be in the range from 10⁷ to 10¹⁰, especially around 2×10⁸. Example 1 shows a fermentation process of the invention where the yeast is not stressed (yeast count of about 10¹⁰ cells per ml). Further guidance in respect of using yeast for fermentation can be found in, e.g., “The alcohol Textbook” (Editors K. Jacques, T. P. Lyons and D. R. Kelsall, Nottingham University Press, United Kingdom 1999.

Enzymes

Cell Wall Degrading Enzymes

According to the invention the cell wall degrading enzyme may be any enzyme capable of degrading the cell wall of the fermenting organism(s) used according to the-invention (i.e., the organism used in the fermentation step of the invention).

Contemplated enzyme activities include: beta-1,3-glucanases, 1,3-beta-glucanases, laminarinases, xylanases, chitinases, mannanases, alpha-1,3-glucanases (mutanase).

Beta-1,3-Glucanases and Laminarinases.

Beta-1,3glucanase includes the group of endo-beta-1,3-glucanases also called laminarinases (E.C. 3.2.1.39 and E.C. 3.2.1.6, Enzyme Nomenclature, Academic Press, Inc, 1992). Pegg et al., Physiol. Plant Pathol., 21, p. 389-409, 1982, showed that a purified endo-beta-1,3-glucanase from tomato in combination with an exo-beta-1,3-glucanase of fungal origin were capable of hydrolysing isolated cell wall of the fungus Verticillium alboatrum. Further, Keen et al., Plant Physiol., 71, p. 460-465 showed that a purified beta-1,3-glucanase from soy bean was capable of degrading isolated cell walls of fungi.

Xylanases

The xylanase activity may be derived from any suitable organism, including fungal and bacterial organisms, such as Aspergillus, Disporotrichum, Penicillium, Neurospora, Fusarium and Trichoderma.

Examples of suitable xylanases include xylanases derived from H. insolens (WO 92/17573; Aspergillus tubigensis (WO 92/01793); A. niger (Shei et al., 1985, Biotech. and Bioeng. Vol. XXVII, pp. 533-538, and Fournier et al., 1985, Biotech. Bioeng. Vol. XXVII, pp. 539-546; WO 91/19782 and EP 463 706); A. aculeatus (WO 94/21785).

In a specific embodiment the xylanase is Xylanase II disclosed in WO 94/21785.

Contemplated commercially available xylanase include SHEARZYME®, BIOFEED WHEAT® (from Novozymes) and SPEZYME® CP (from Genencor Int., USA).

Chitinases

Chitinases include the groups of exo-chitinases and endochitinases. Exochitinases are also referred to as chitobiosidases or beta-N-acetylhexosaminidases (E.C. 3.2.1.52, Enzyme Nomenclature, Academic Press, Inc., 1992). Endochitinases (E.C. 3.4.1.14) are enzymes, which randomly hydrolyse N-acetyl-beta-D-glucosaminide 1,4-beta-linkages of chitin and chitodextrins.

Fungal chitinases include the ones described by Harman et al., (1993), Mol. Plant Pathology 83, 313-318; Blaiseau and Lafay, (1992), Elsevier science publisher B.V., 243-248; and Gracia, (1994), Current Genetics 27, 83-89. Also contemplated chitinases include the ones described in WO 92122314 (Cornell Research Foundation, INC) describes two chitinases from Trichoderma harzianum P1 (ATCC 74058); WO 94/24288 and WO 94/02598 (Cornell Research Foundation, INC) disclosing two chitinases from Trichoderma harzianum P1 (ATCC 74058); and EP 440.304 which concerns plants exhibiting a relative overexpression of at least one gene encoding intracellular chitinase and intra- or extracellular beta-1,3 glucanase.

Mannanases

Mannanases have been identified in several Bacillus organisms. For example, Talbot et al., Appl. Environ. Microbiol., Vol.56, No. 11, pp. 3505-3510 (1990) describes a beta-mannanase derived from Bacillus stearothermophilus in dimer form having molecular weight of 162 kDa and an optimum pH of 5.5-7.5. Mendoza et al., World J. Microbiol. Biotech., Vol. 10, No. 5, pp. 551-555 (1994) describes a beta-mannanase derived from Bacillus subtilis having a molecular weight of 38 kDa, an optimum activity at pH 5.0 and 55° C. and a pi of 4.8. JP-03047076 discloses a beta-mannanase derived from Bacillus sp., having a molecular weight of 37±3 kDa measured by gel filtration, an optimum pH of 8-10 and a pI of 5.3-5.4. JP-63056289 describes the production of an alkaline, thermostable beta-mannanase which hydrolyses beta-1,4-D-mannopyranoside bonds of e.g. mannans and produces manno-oligosaccharides. JP-63036774 relates to the Bacillus microorganism FERM P-8856, which produces beta-mannanase and beta-mannosidase at an alkaline pH. JP-08051975 discloses alkaline beta-mannanases from alkalophilic Bacillus sp. AM-001. A purified mannanase from Bacillus amyloliquefaciens useful in the bleaching of pulp and paper and a method of preparation thereof is disclosed in WO 97/11164. WO 94/25576 discloses an enzyme from Aspergillus aculeatus, CBS 101.43, exhibiting mannanase activity, which may be useful for degradation or modification of plant or algae cell wall material. WO 93/24622 discloses a mannanase isolated from Trichoderma reseei useful for bleaching lignocellulosic pulps.

Mutanases

Mutanases are alpha-1,3-glucanases (also known as α-1,3-glucanohydrolases), which degrade the alpha-1,3-glycosidic linkages in mutan. Mutanases have been described from two species of Trichoderma (Hasegawa et al., (1969), Journal of Biological Chemistry 244, p. 5460-5470; Guggenheim and Haller, (1972), Journal of Dental Research 51, p. 394-402) and from a strain of Streptomyces (Takehara et al., (1981), Journal of Bacteriology 145, p. 729-735), Cladosporium resinae (Hare et al. (1978), Carbohydrate Research 66, p. 245-264), Pseudomonas sp. (U.S. Pat. No. 4,438,093), Flavobacterium sp. (JP 77038113), Bacillus circulanse (JP 63301788) and Aspergillus sp. A mutanase gene from Trichoderma harzianum has been cloned and sequenced (Japanese Patent No. 4-58889-A from Nissin Shokuhin Kaisha LDT). A preferred mutanase is described in WO 98/00528 (from Novozymes).

Preferred cell wall degrading enzymes have an optimum activity within the pH and temperature of the fermentation step, i.e., at acidic pH, in particular at a pH between 3-6, preferably between pH 4-5 and a temperature between 26-34° C., in particular about 32° C.

Carbohydrate-Source Generating Enzymes

Carbohydrate-source generating enzymes include any enzyme capable of generating a carbohydrate source, which the fermenting organism can use as energy source, for the fermentation or directly or indirectly converting into the desired fermentation product.

Specifically contemplated carbohydrate-source generating enzymes are glucoamylase, beta-amylase, and maltogenic amylase.

Glucoamylase

The saccharification step (c) or a combined saccharification and fermentation step (SSF step) may be carried out in the presence of a glucoamylase derived from a microorganism or a plant. Preferred is glucoamylase of fungal or bacterial origin selected from the group consisting of Aspergillus glucoamylases, in particular A. niger G1 or G2 glucoamylase (Boel et al. (1984), EMBO J. 3 (5), p. 1097-1102), or variants thereof, such as disclosed in WO 92/00381 and WO 00/04136; the A. awamori glucoamylase (WO 84/02921), A. oryzae (Agric. Biol. Chem. (1991), 55 (4), p. 941-949), or variants or fragments thereof.

Other contemplated Aspergillus glucoamylase variants include variants to enhance the thermal stability: G137A and G139A (Chen et al. (1996), Prot Engng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Furthermore Clark Ford presented a paper on Oct. 17, 1997, ENZYME ENGINEERING 14, Beijing/China Oct. 12-17, 1997, Abstract number: Abstract book p. 0-61. The abstract suggests mutations in positions G137A, N20C/A27C, and S30P in an Aspergillus awamori glucoamylase to improve the thermal stability. Other glucoamylases include Talaromyces glucoamylases, in particular derived from Talaromyces emersonii (WO 99/28448), Talaromyces leycettanus, Talaromyces duponti (U.S. Pat. No. 32,153), Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

Glucoamylases may in an embodiment be added in an amount of 0.02-2 AGU/g DS, preferably 0.1-1 AGU/g DS, such as 0.2 AGU/g DS

The ratio between acidic fungal alpha-amylase activity (AFAU) per glucoamylase activity (AGU) (AFAU per AGU) may in one embodiment be at least 0.1, in particular at least 0.16, such as in the range from 0.12 to 0.30.

Commercial products include AMG 200L; AMG 300 L; SAN™ SUPER and AMG™ E (from Novozymes); OPTIDEX™ 300 (from Genencor Int.); AMIGASE™ and AMIGASE™ PLUS (from DSM); G-ZYME™ G900 (from Enzyme Bio-Systems); G-ZYME™ G990 ZR (A. niger glucoamylase and low protease content).

Beta-Amylase

Beta-amylase (E.C 3.2.1.2) is the name traditionally given to exo-acting maltogenic amylases, which catalyze the hydrolysis of 1,4-alpha-glucosidic linkages in amylose, amylopectin and related glucose polymers. Maltose units are successively removed from the non-reducing chain ends in a step-wise manner until the molecule is degraded or, in the case of amylopectin, until a branch point is reached. The maltose released has the beta anomeric configuration, hence the name beta-amylase.

Beta-amylases have been isolated from various plants and microorganisms (W. M. Fogarty and C. T. Kelly, Progress in Industrial Microbiology, vol. 15, pp. 112-115, 1979). These beta-amylases are characterized by having optimum temperatures in the range from 40° C. to 65° C. and optimum pH in the range from 4.5 to 7. A commercially available beta-amylase from barley is SPEZYME™ BBA 1500 from Genencor Int., USA.

Maltogenic Amylase

Maltogenic amylases (glucan 1,4-alpha-maltohydrolase, E.C. 3.2.1.133) are able to hydrolyse amylose and amylopectin to maltose in the alpha-configuration. Furthermore, a maltogenic amylase is able to hydrolyse maltotriose as well as cyclodextrin. A specifically contemplated maltogenic amylase includes the one disclosed in EP patent no.120,693 derived from Bacillus stearothermophilus C599. A commercially available maltogenic amylase is MALTOGENASE™ from Novozymes A/S

Phytase

The phytase used according to the invention may be any enzyme capable of effecting the liberation of inorganic phosphate from phytic acid (myo-inositol hexakisphosphate) or from any salt thereof (phytates). Phytases can be classified according to their specificity in the initial hydrolysis step, viz. according to which phosphate-ester group is hydrolyzed first. The phytase to be used in the invention may have any specificity, e.g., be a 3-phytase (EC 3.1.3.8), a 6-phytase (EC 3.1.3.26) or a 5-phytase (no EC number).

In a preferred embodiment the phytase has a temperature optimum in the range from 25-70° C., preferably 28-50° C., especially 30-40° C. This is advantageous when the phytase is added during fermentation.

In another preferred embodiment the phytase has a temperature optimum above 50° C., such as in the range from 50-70° C. This is advantageous when the phytase is added during pre-saccharification. A preferred suitable dosage of the phytase is in the range from 0.005-25 FYT/g DS, preferably 0.01-10 FYT/g, such as 0.1-1 FYT/g DS.

Here, the phytase activity is determined FYT units, one FYT being the amount of enzyme that liberates 1 micromole inorganic ortho-phosphate per min. under the following conditions: pH 5.5; temperature 37° C.; substrate: sodium phytate (C₆H₆O₂₄P₆Na₁₂) at a concentration of 0.0050 mole/l.

The phytase may be derived from plants or microorganisms, such as bacteria or fungi, e.g., yeast or filamentous fungi.

The plant phytase may be from wheat-bran, maize, soy bean or lily pollen. Suitable plant phytases are described in Thomlinson et al, Biochemistry, 1 (1962), 166-171; Barrientos et al, Plant. Physiol., 106 (1994), 1489-1495; WO 98/05785; WO 98/20139.

A bacterial phytase may be from genus Bacillus, Pseudomonas or Escherichia, specifically the species B. subtilis or E. coli. Suitable bacterial phytases are described in Paver and Jagannathan, 1982, Journal of Bacteriology 151:1102-1108; Cosgrove, 1970, Australian Journal of Biological Sciences 23:1207-1220; Greiner et al, Arch. Biochem. Biophys., 303, 107-113, 1993; WO 98/06856; WO 97/33976; WO 97/48812.

A yeast phytase or myo-inositol monophosphatase may be derived from genus Saccharomyces or Schwanniomyces, specifically species Saccharomyces cerevisiae or Schwanniomyces occidentalis. The former enzyme has been described as a Suitable yeast phytases are described in Nayini et al, 1984, Lebensmittel Wissenschaft und Technologie 17:24-26; Wodzinski et al. Adv. Appl. Microbiol., 42, 263-303; AU-A-24840/95;

Phytases from filamentous fungi may be derived from the fungal phylum of Ascomycota (ascomycetes) or the phylum Basidiomycota, e.g., the genus Aspergillus, Thermomyces (also called Humicola), Myceliophthora, Manascus, Penicillium, Peniophora, Agrocybe, Paxillus, or Trametes, specifically the species Aspergillus terreus, Aspergillus niger, Aspergillus niger var. awamori, Aspergillus ficuum, Aspergillus fumigatus, Aspergillus oryzae, T. lanuginosus (also known as H. lanuginosa), Myceliophthora thermophila, Peniophora lycii, Agrocybe pediades, Manascus anka, Paxillus involtus, or Trametes pubescens. Suitable fungal phytases are described in Yamada et al., 1986, Agric. Biol. Chem. 322:1275-1282; Piddington et al., 1993, Gene 133:55-62; EP 684,313; EP 0 420 358; EP 0 684 313; WO 98/28408; WO 98/28409; JP 7-67635; WO 98/44125; WO 97/38096; WO 98/13480.

Modified phytases or phytase variants are obtainable by methods known in the art, in particular by the methods disclosed in EP 897010; EP 897985; WO 99/49022; WO 99/48330.

Commercially available phytases contemplated according to the invention include BIOFEED PHYTASE™, PHYTASE NOVO™ CT or L (all available from Novozymes A/S), or NATUPHOS™ NG 5000 (available from DSM).

Alpha-Amylases

The liquefaction step may be performed in the presence of an alpha-amylase derived from a microorganism or a plant. Preferred alpha-amylases are of fungal or bacterial origin. Bacillus alpha-amylases (often referred to as “Termamyl-like alpha-amylases”), variant and hybrids thereof, are specifically contemplated according to the invention. Well-known Termamyl-like alpha-amylases include alpha-amylase derived from a strain of B. licheniformis (commercially available as Termamyl™), B. amyloliquefaciens, and B. stearothermophilus alpha-amylase (BSG). Other Termamyl-like alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31. In the context of the present invention a Termamyl-like alpha-amylase is an alpha-amylase as defined in WO 99/19467 on page 3, line 18 to page 6, line 27. Contemplated variants and hybrids are described in WO 96/23874, WO 97/41213, and WO 99/19467. Contemplated alpha-amylase derived from a strain of Aspergillus includes Aspergillus oryzae and Aspergillus niger—amylases.

Commercially available alpha-amylase products and products containing alpha-amylases include TERMAMYL™ SC, FUNGAMYL™, LIQUOZYME™ and SAN™ SUPER.

Fungal alpha-amylases may be added in an amount of 0.001-1.0 AFAU/g DS, preferably from 0.002-0.5 AFAU/g DS, preferably 0.02-0.1 AFAU/g DS.

Bacillus alpha-amylases may be added in effective amounts well known to the person skilled in the art.

Proteases

The higher fermentation efficiency obtained when using protease(s) has generally been attributed, to an increase in the FAN (Free amino nitrogen) level and thereby an increase in the rate of metabolism of the yeast. Proteases, in particular acidic proteases, including especially Rhizomucor miehei protease, has according to the invention been demonstrated to reduce flocculation of yeast cells and attachment of yeast cells to insoluble material in high gravity fermentation of ethanol. This reduced flocculation itself will result in higher fermentation efficiency (productivity) and a higher immediate alcohol yield.

Suitable proteases include fungal and bacterial proteases. Preferred proteases are acidic proteases, i.e., proteases characterized by the ability to hydrolyze proteins under acidic conditions below pH 7.

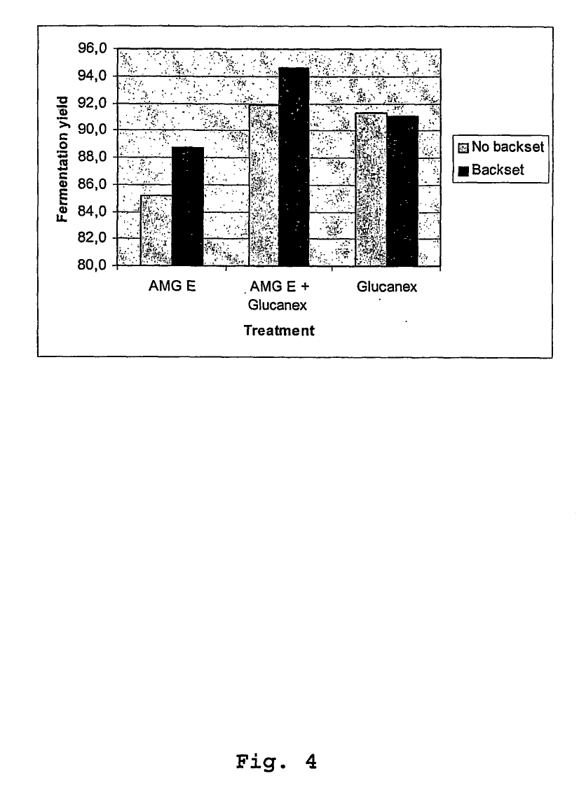

Suitable acid fungal proteases include fungal proteases derived from Aspergillus, Rhizomucor, Rhizopus, Candida, Coriolus, Endothia, Enthomophtra, Irpex, Penicillium, Sclerotium and Torulopsis. Especially contemplated are proteases derived from Aspergillus niger (see, e.g., Koaze et al., (1964), Agr. Biol. Chem. Japan, 28, 216), Aspergillus saitoi (see, e.g., Yoshida, (1954) J. Agr. Chem. Soc. Japan, 28, 66), Aspergillus awamori (Hayashida et al., (1977) Agric. Biol. Chem., 42(5), 927-933, Aspergillus aculeatus (WO 95/02044), or Aspergillus oryzae, such as the pepA protease; and acidic proteases from Rhizoucor pusillus (Mucor pusillus), Rhizomucor miehei (Mucor miehei). A preferred embodiment of the invention includes the use of Rhizomucor miehei (Mucor miehei) acidic protease encoded by the sequence shown in FIGS. 4a-4b in EP238023. Another preferred embodiment comprises the use of R. miehei protease produced in Aspergillus oryzae as described in Example 7 in EP238023. A third preferred embodiment comprises the use of a fermentation product from transformed A. oryzae in which the R. miehei protease is co-expressed together with the native A. oryzae protease. The R. miehei protease may according to the invention preferably be added in amounts of 0.01-1.0 mg enzyme protein per g of dry substance in the fermenting medium, preferably 0.1-0.5 mg enzyme protein per 9 of dry substance in the fermenting medium. The use of protease, in particular acidic proteases, especially Rhizomucor miehei protease may according to the invention be applicable to batch as well as to continued fermentation processes.

Bacterial proteases, which are not acidic proteases, such as Bacillus proteases, include the commercially available products ALCALASE® and NEUTRASE® (available from Novozymes A/S).

ALCALASE™ is a Bacillus licheniformis protease (subtilisin, Carlsberg). ALCALASE™ may according to the invention preferably be added is amounts of 10⁻⁷ to 10⁻³ gram active protease protein/g DS, in particular 10⁻⁶ to 10⁻⁴ gram active protease protein/g DS, or in amounts of 0.1-0.0001 AU/g DS, preferably 0.00025-0.001 AU/g DS.

FLAVOURZYME™ is a protease preparation derived from Aspergillus oryzae. FLAVOURZYME™ may according to the invention preferably be added in amounts of 0.01-1.0 LAPU/g DS, preferably 0.05-0.5 LAPU/g DS)

In general protease(s) may in one embodiment be added in an amount of 10⁻⁷ to 10⁻³ gram active protease protein/g DS, in particular 10⁻⁶ to 10⁻⁴ gram active protease protein/g DS

Debranching Enzymes

Pullulanase

Contemplated pullulanases include the thermostable pullulanase from, e.g., Pyrococcus or Bacillus sp., including protein engineered pullulanases from, e.g., a Bacillus strain such as Bacillus acidopullulyticus (e.g, the one described in FEMS Mic. Let. (1994)115, 97-106), Bacillus deramificans (e.g., the Bacillus deramificans pullulanase with GeneBank accession number Q68699), or Bacillus naganoensis.

Contemplated commercially available pullulanases include PROMOZYME™ D and PROMOZYME™ 200 L and 400 L, from Novozymes; OPTIMAX™ 3000 is a pullulanase derived from Bacillus sp. (from Genencor Int.); ULTRADEX™ which is a pullulanase from Bacillus naganoensis from Enzyme Bio-Systems.

Glucoamylase/Pullulanase Combination Products

Further, commercially available combination products include DEXTROZYME™ E and DEXTROZYME™ E ULTRA, comprising glucoamylase from A. niger and pullulanase (from Bacillus); DEXTROZYME™ D which is a balanced mixture of glucoamylase derived from Aspergillus niger and a pullulanase; DEXTROZYME™ 225/75 L which is a balanced mixture of glucoamylase derived from Aspergillus niger and pullulanase from Novozymes; OPTIMAX™ 7525 which is a blend of glucoamylase and heat stable pullulanase.

According to the invention the ration between glucoamylase and pullulanase determined as, e.g., AGU/PUN may be from 5:1 to 1:5, preferably 4:1 to 1:4, such as 1:1 or 2:1 or 3:1.

Isoamylase

Contemplated isoamylase according to the invention include Pseudomonas amyloderamosa Biochim. Biophys. Acta, 1087, p.309-315 (1990); Pseudomonas sp. (EP 0 302 838 A2); Flavobacterium sp. (WO 96/03513), in particular Flavobacterium sp. IFO 14590 (shown as SEQ ID NO: 11 in WO 99/01545); Flavobacterium odoratum (JP08023981 -A); Sulfolobus acidocaldarius Biochim. Biophys. Acta, 1291, p.177-181 (1996); Rhodothermus marinus (WO 99/01545), in particular Rhodothermus marinus DSM 4252 (shown as SEQ ID NO: 4 in WO 99/01545); Flavobacterium devorans ATCC 10829 (shown as SEQ ID NO: 12 in WO 99/01545); Xanthomonas campestris ATCC 31922 (shown as SEQ ID NO: 13 in WO 99/01545); Rhodothermus obamensis JCM 9785 (shown as SEQ ID NO: 14 in WO 99/01545).

Composition

Finally the invention related to a composition comprising all of the following enzyme activities: carbohydrate-source generating enzyme activity (as defined), alpha-amylase activity, protease activity and debranching enzyme activity (as defined). The composition may further comprise a phytase and/or protease, in particular an acid protease, such as an acid fungal protease.

Materials and Methods

Enzymes:

Beta-glucanase preparation derived from Trichoderma: GLUCANEX™ (available from Novozymes) (SE-2000-0247, 183 BGXU/g)

Glucoamylase and acid alpha-amylase: Aspergillus niger glucoamylase (available as AMG E from Novozymes A/S) (SE-2000-00034, 382 AGU/g).

Protease; FLAVOURZYME™ is a protease/peptidase complex derived from Aspergillus oryzae (Available from Novozymes A/S).

Protease derived from Rhizomucor miehei was produced in Aspergillus oryzae as described in Example 7 in EP 238023. An experimental enzyme product with an enzyme protein content of 48.8 mg/g was prepared.

Pullulanase; PROMOZYME® is derived from Bacillus acidopullulyticus and described in EP 63.909 (available from Novozymes).

Alpha-amylase: BSG (B. stearothermophilus alpha-amylase which is available from. Novozymes as TERMAMYL™ SC).

Substrate and Yeast:

Liquefied whole corn mash: Liquefied whole corn mash was prepared by a hot slurry process and Termamyl SC treatment. The mash has a DE of about 17 and a dry substance of about 28% (SS-99-00007).

Liquefied whole corn mash: Liquefied whole corn mash was prepared by a hot slurry process and Termamyl SC treatment. The mash has a DE of about 12 and a dry substance of about 30% (SS-00-00012).

Further reference on how to produce industrial whole corn mash is given in “The Alcohol Textbook” Editors. T. P. Lyons, D. R. Kesall and J. E. Murtagh. Nottingham University Press 1995.

The yeast applied was a Saccharomyces cervisiae (S-00640-2)

Determination of Beta-Glucanase Units

One Beta Glucanase Unit (BGXU) corresponds to the quantity of enzyme required to produce 1 micromole of reducing sugars per minute under standard conditions. A detailed description of Novozymes's analytic method is available on request.

Determination of Alpha-Amylase Activity (KNU)

1. Phadebas Assay

Alpha-amylase activity is determined by a method employing Phadebas® tablets as substrate. Phadebas tablets (Phadebas® Amylase Test, supplied by Pharmacia Diagnostic) contain a cross-linked insoluble blue-colored starch polymer, which has been mixed with bovine serum albumin and a buffer substance and tabletted.

For every single measurement one tablet is suspended in a tube containing 5 ml 50 mM Britton-Robinson buffer (50 mM acetic acid, 50 mM phosphoric acid, 50 mM boric acid, 0.1 mM CaCl₂, pH adjusted to the value of interest with NaOH). The test is performed in a water bath at the temperature of interest. The alpha-amylase to be tested is diluted in x ml of 50 mM Britton-Robinson buffer. 1 ml of this alpha-amylase solution is added to the 5 ml 50 mM Britton-Robinson buffer. The starch is hydrolyzed by the alpha-amylase giving soluble blue fragments. The absorbance of the resulting blue solution, measured spectrophotometrically at 620 nm, is a function of the alpha-amylase activity.

It is important that the measured 620 nm absorbance after 10 or 15 minutes of incubation (testing time) is in the range of 0.2 to 2.0 absorbance units at 620 nm. In this absorbance range there is linearity between activity and absorbance (Lambert-Beer law). The dilution of the enzyme must therefore be adjusted to fit this criterion. Under a specified set of conditions (temperature, pH, reaction time, buffer conditions) 1 mg of a given alpha-amylase will hydrolyze a certain amount of substrate and a blue colour will be produced. The colour intensity is measured at 620 nm. The measured absorbance is directly proportional to the specific activity (activity/mg of pure alpha-amylase protein) of the alpha-amylase in question under the given set of conditions.

2. Alternative Method

Alpha-amylase activity is determined by a method employing the PNP-G7 substrate. PNP-G7 which is a abbreviation for p-nitrophenyl-alpha,D-maltoheptaoside is a blocked oligosaccharide which can be cleaved by an endo-amylase. Following the cleavage, the alpha-Glucosidase included in the kit digest the substrate to liberate a free PNP molecule which has a yellow colour and thus can be measured by visible spectophometry at Lambda=405 nm. (400-420 nm). Kits containing PNP-G7 substrate and alpha-Glucosidase is manufactured by Boehringer-Mannheim (cat. No. 1054635).

To prepare the substrate one bottle of substrate (BM 1442309) is added to 5 ml buffer (BM1442309). To prepare the alpha-Glucosidase one bottle of alpha-Glucosidase (BM 1462309) is added to 45 ml buffer (BM1442309). The working solution is made by mixing 5 ml alpha-Glucosidase solution with 0.5 ml substrate.

The assay is performed by transforming 20 micro I enzyme solution to a 96 well microtitre plate and incubating at 25° C. 200 micro I working solution, 25° C. is added. The solution is mixed and pre-incubated 1 minute and absorption is measured every 15 sec. over 3 minutes at OD 405 nm.

The slope of the time dependent absorption-curve is directly proportional to the specific activity (activity per mg enzyme) of the alpha-amylase in question under the given set of conditions.

Determination of Acid Amylolytic Activity (FAU)

One Fungal Alpha-Amylase Unit (1 FAU) is defined as the amount of enzyme, which breaks down 5.26 g starch (Merck Amylum solubile Erg. B.6, Batch 9947275) per hour at Novozymes standard method for determination of alpha-amylase based upon the following standard conditions:

| | |
|---|---|
| Substrate | Soluble starch |
| Temperature | 37° C. |
| pH | 4.7 |
| Reaction time | 7-20 minutes |

A detailed description of Novozymes' method is available on request.

Determination of Acid Alpha-Amylase Activity (AFAU)

Acid alpha-amylase activity is measured in AFAU (Acid Fungal Alpha-amylase Units), which are determined relative to an enzyme standard.

The standard used is AMG 300 L (wild type A. niger G1 AMG sold by Novozymes). The neutral alpha-amylase in this AMG falls after storage at room temperature for 3 weeks from approx. 1 FAU/mL to below 0.05 FAU/mL.

The acid alpha-amylase activity in this AMG standard is determined in accordance with AF 9⅓ (available from Novozymes A/S method for the determination of fungal alpha-amylase). In this method, 1 AFAU is defined as the amount of enzyme, which degrades 5.260 mg starch dry matter per hour under standard conditions. Iodine forms a blue complex with starch but not with its degradation products. The intensity of colour is therefore directly proportional to the concentration of starch. Amylase activity is determined using reverse colorimetry as a reduction in the concentration of starch under specified analytic conditions.

| | | |
|---|---|---|
| | Alpha-amylase | |
| Starch + Iodine | → | Dextrins + Oligosaccharides |
| | 40° C., pH 2.5 | |
| Blue/violet | t = 23 sec. | Decolouration |

Standard Conditions/Reaction Conditions: (Per Minute)

| Substrate: | starch, approx. 0.17 | g/L |
|---|---|---|
| Buffer: | Citrate, approx. 0.03 | M |
| Iodine (I₂): | 0.03 | g/L |
| CaCl₂: | 1.85 | mM |
| pH: | 2.50 + | 0.05 |
| Incubation temperature: | 40° | C. |
| Reaction time: | 23 | seconds |
| Wavelength: | Lambda = 590 | nm |
| Enzyme concentration: | 0.025 | AFAU/mL |
| Enzyme working range: | 0.01-0.04 | AFAU/mL |

Further details can be found in EB-SM-0259.02/01 available on request from Novozymes, and hereby incorporated by reference.

Determination of Glucoamylase Activity (AGU)

The Novo Amyloglucosidase Unit (AGU) is defined as the amount of enzyme, which hydrolyzes 1 micromole maltose per minute at 37° C. and pH 4.3. A detailed description of the analytical method (AEL-SM-0131) is available on request from Novozymes.

The activity is determined as AGU/ml by a method modified after (AEL-SM-0131, available on request from Novozymes) using the Glucose GOD-Perid kit from Boehringer Mannheim, 124036. Standard: AMG-standard, batch 7-1195, 195 AGU/ml. 375 microL substrate (1% maltose in 50 mM Sodium acetate, pH 4.3) is incubated 5 minutes at 37° C. 25 microL enzyme diluted in sodium acetate is added. The reaction is stopped after 10 minutes by adding 100 microL 0.25 M NaOH. 20 microL is transferred to a 96 well microtitre plate and 200 microL GOD-Perid solution (124036, Boehringer Mannheim) is added. After 30 minutes at room temperature, the absorbance is measured at 650 nm and the activity calculated in AGU/ml from the AMG-standard.

Proteolytic Activity—Anson Units (AU)

The proteolytic activity may be determined with denatured hemoglobin as substrate. In the Anson-Hemoglobin method for the determination of proteolytic activity denatured hemoglobin is digested, and the undigested hemoglobin is precipitated with trichloroacetic acid (TCA). The amount of TCA soluble product is determined with phenol reagent, which gives a blue color with tyrosine and tryptophan.

One Anson Unit (AU) is defined as the amount of enzyme, which under standard conditions (i.e. 25° C., pH 7.5 and 10 min. reaction time) digests hemoglobin at an initial rate such that there is liberated per minute an amount of TCA soluble product, which gives the same color with phenol reagent as one milliequivalent of tyrosine.

A folder AF 4/5 describing the analytical method in more detail is available upon request to Novozymes A/S, Denmark, which folder is hereby included by reference.

Protease Assay Methods (LAPU)

1 Leucine Amino Peptidase Unit (LAPU) is the amount of enzyme which decomposes 1 microM substrate per minute at the following conditions: 26 mM of L-leucine-p-nitroanilide as substrate, 0.1 M Tris buffer (pH 8.0), 40° C., 10 minutes reaction time.

Beta-Amylase Activity (DP°)

The activity of SPEZYME® BBA 1500 is expressed in Degree of Diastatic Power (DP°). It is the amount of enzyme contained in 0.1 ml of a 5% solution of the sample enzyme preparation that will produce sufficient reducing sugars to reduce 5 ml of Fehling's solution when the sample is incubated with 100 ml of substrate for 1 hour at 20° C.

Pullulanase Activity (New Pullulanase Unit Novo (NPUN)

One new Pullulanase Unit Novo (NPUN) is a unit of endo-pullulanase activity and is measured relative to Novozymes standard made on 0.7% Red Pullulan, 40° C., pH 4.5, 30 minutes reaction time. A detailed description of the analysis method is available on request Novozymes A/S, Denmark (SOP No.: EB-SM.0420.02/01).

Pullulanase Activity (PUN)

Pullulanase activity may be determined relative to a pullulan substrate. Pullulan is a linear D-glucose polymer consisting essentially of maltotriosyl units joined by 1,6-alpha-links. Endopullulanases hydrolyze the 1,6-alpha-links at random, releasing maltotriose, 6³-alpha-maltotriosyl-maltotriose, 6³-alpha-(6³-alpha-maltotriosyl-maltotriosyl)-maltotriose, etc. the number of links hydrolyzed is determined as reducing carbohydrate using a modified Somogyi-Nelson method.

One pullulanase unit (PUN) is the amount of enzyme which, under standard conditions (i.e. after 30 minutes reaction time at 40° C. and pH 5.0; and with 0.2% pullulan as substrate) hydrolyzes pullulan, liberating reducing carbohydrate with a reducing power equivalent to 1 micro mol glucose per minute.

A folder, AF 190/2-GB, describing this analytical method in more detail is available upon request to Novozymes A/S, Denmark, which folder is hereby included by reference.

Assessment of Flocculation

Flocculation is measured according to D'Hautcourt & Smart, Journal of the American Society of Brewing Chemists Vol. 57: 123-128 (1999). The procedure is the following: Washing of pure yeast with water or 250 mM NaCl (physiological salt solution), addition of Ca++, enzyme treatment, sedimentation for 60 minutes followed by measurement of sediment volume

Assessment of Ethanol Production and Fermentation Efficiency

During the fermentation basically three products are made; ethanol, CO₂ and biomass. It is generally known that based on the metabolic conversion rate the amount of ethanol produced can be calculated from the CO₂ produced.

The relationship between amount of CO₂ loss and the weight of ethanol is calculated as CO₂ loss (g)×1.045=EtOH (g).

### EXAMPLES

### Example 1

This example demonstrates how the presence of R. miehei protease reduces flocculation.

2.5 g of washed yeast was suspended in 100 mL of ion-exchanged water at room temperature. The suspension was stirred on a magnetic stirrer for 15 minutes. 15 mL samples were transferred to centrifuge tubes with volume indication. NaCl, CaCl₂ and R. Miehei protease was added to create the solutions given in table 1. Incubation of solutions was made at room temperature for 15 minutes in a rotary shaker, which turned the closed tubes end-over-end at 20 rpm. Hereafter the tubes were left in vertical position for 60 minutes after which the volume of the sediment was measured. The results are shown in table 1.

TABLE 1
Effect of Rhizomucor miehei on volume of sediment.
Enzyme concentration was 0.03 mg enzyme protein/mL.

| Test solutions | mL sediment |
|---|---|
| 250 mM NaCl | 0.165 |
| 4 mM CaCl₂ | 0.245 |
| 4 mM CaCl₂ + R. Miehei protease | 0.194 |

### Example 2

This example demonstrates how the presence of R. miehei protease increases fermentation rate and ethanol yield.

For propagation of the yeast 600 g mash was transferred to a 1 L Erlenmeyer flask and 400 g of water was added, resulting in approximately 20% dry substance in the propagation mash. pH was adjusted to 6.0 in the mash using NaOH. 2.0 g urea, Aspergillus niger glucoamylase (AMG E, Novozymes AIS) was dosed at 0.3 AGU/g of dry matter and 0.48 g dry yeast was added. The flask was then incubated in a water bath at 32° C. for 4 hours under constant stirring at minimum 200 rpm.

The number of yeast cells in the propagate was measured using a Fuchs-Rosenthal Haemocytometer and a yeast count of approximately 60 million viable yeast cells per mL was obtained.

A simultaneous saccharification and fermentation process was carried out the following way: pH was adjusted to 4.5 in the mash. A dry matter determination was made and adjustment was made to 33.0 % (w/w dry matter). Aspergilius niger glucoamylase was dosed at 0.4 AGU/g of dry matter. 240 g of mash was filled into a 500 mL blue cap bottle. pH of the mash was re-adjusted to 4.5. The two treatments were added respectively 0 and 6.8 mg R. miehei protease enzyme protein/kg dry matter of the mash. 10 g of propagated yeast was added to each blue cap bottle, time was set to t=0 and the bottles were closed with a yeast-lock filled with corn oil. All bottles were incubated in a shaking water bath preset at 32° C. The bottles were weighed at t=0 and at regular intervals until 72 hours for measuring CO₂ weight loss during the fermentation progress. The results are shown in table 2.

TABLE 2
Weight loss (g) during fermentations with and
without R. miehei protease added. AMG E was
used as 0.4 AGU/g dry matter and R. miehei protease
6.8 mg enzyme protein/kg dry matter of the mash

| | | AMG E + |
|---|---|---|
| Hours | AMG E | R. mieheiprotease |
| 0.00 | 0.00 | 0.00 |
| 1.33 | 0.00 | 0.00 |
| 2.67 | 0.09 | 0.13 |
| 18.58 | 7.20 | 7.38 |
| 24.20 | 12.12 | 12.67 |
| 28.25 | 14.98 | 15.65 |
| 42.58 | 21.73 | 22.53 |
| 48.00 | 23.04 | 23.70 |
| 52.50 | 23.62 | 24.18 |
| 66.50 | 24.15 | 24.64 |
| 72.00 | 24.19 | 24.70 |

### Example 3

Fermentation with Glucoamylase and Trichoderma Preparation

250 mL of liquefied whole corn mash (DE12 and DE17, respectively) was filled into a 500 mL blue cap bottles. The DE 12 mash viscosity was reduced by adding 50 mL of milliQ water to make it possible to use magnetic stirrers. The pH of the mash was adjusted to 4.5. Before the actual fermentation a pre-saccharification was carried out by adding glucoamylase (AMG E: 0.2 AG/g dry matter) and placing the bottles in a water bath at 60° C. for 70 minutes. The bottles were cooled in a water bath for 40 minutes to 30° C. and dry yeast was added at a dosage of 0.8 g/bottle (in order to reach 30° C. within 40 minutes it was necessary to add ice to the water bath). The dry yeast was added in excess, meaning that the yeast nutrition and viability is the limiting factor for the fermentation rate. The bottles were closed using a yeast-lock filled with concentrated H₂SO₄. The fermentation was continued for 72 hours and by weighing the bottle at regular intervals the CO₂ loss was monitored. Every 24, 48 and 72 hours samples for HPLC analysis were taken out.

Treatments

The following treatments were made for each mash:

AMG E: 0.2 AG/g dry matter

AMG E: 0.2 AG/g dry matter+GLUCANEX™: 2,4 BGXU/g dry matter

GLUCANEX™: 2.4 BGXU/g dry matter

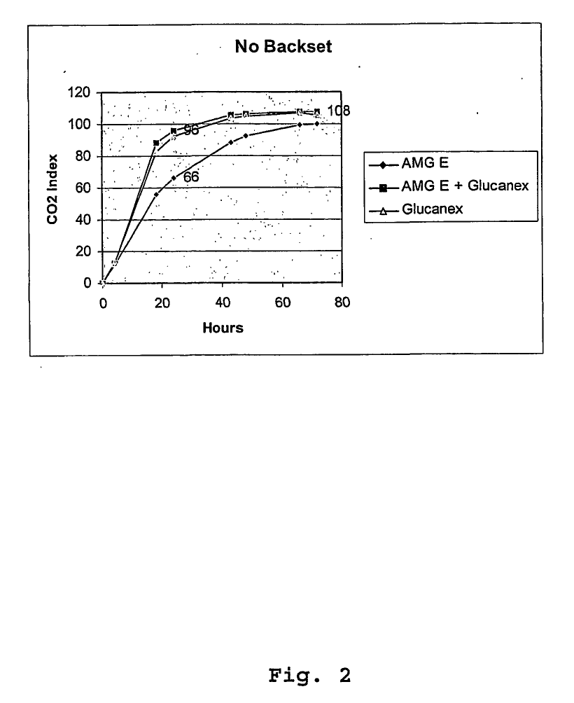

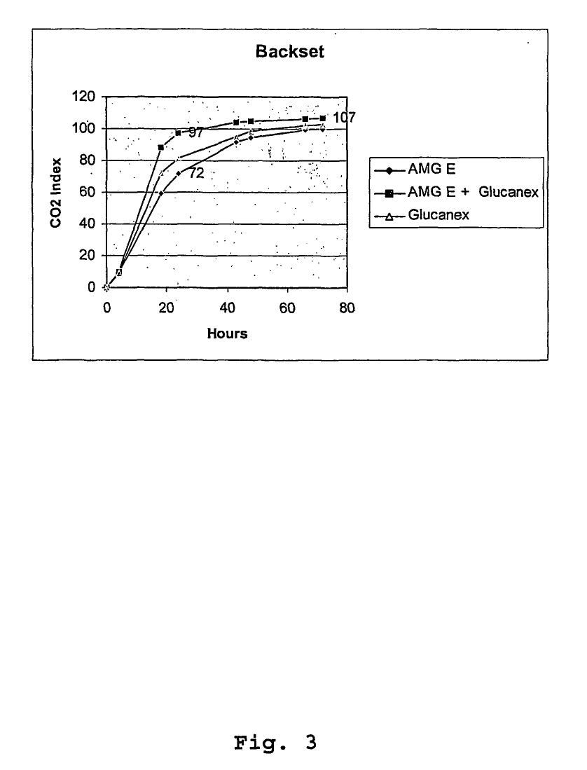

Each treatment was done in triplicates. The CO₂ development as a function of time is shown in FIGS. 2 and 3. Note that the Figures are indexed with AMG E only at 72 hours as index 100. Index labels are shown for AMG E+GLUCANEX™ at 24 hours and AMG E+GLUCANEX™ at 72 hours. There was a high correlation between CO₂ weight loss and ethanol concentration.

As can be seen GLUCANEX™ had a positive effect on the fermentation rate and ethanol yields.

1. A process of producing a fermentation product, which process comprises a fermentation step, wherein the fermentation step is carried out in the presence of at least one carbohyrate-source generating-enzyme activity and at least one alpha-amylase activity.

2. The process of claim 1, wherein further a protease is present.

3. The process of claim 1 or 2, wherein further a debranching enzyme activity is present.

4. The process of claims 1-3, wherein the carbohydrate-source generating enzyme is a glucoamylase, in particular derived from Aspergillus niger or Talaromyces emersonli; or beta-amylase, in particular derived from barley; or a maltogenic amylase, in particular derived from Bacillus stearothermophilus.

5. The process of claims 1-4, wherein the alpha-amylase is an acid alpha-amylase, in particular an acid fungal alpha-amylase, such as an acid fungal alpha-amylase derived from Aspergillus niger or Aspergillus oryzae.

6. The process of claims 1-5, wherein the ratio between acid fungal alpha-amylase activity (AFAU) per glucoamylase activity (AGU) (AFAU per AGU) is at least 0.1, in particular at least 0.16, especially in the range from 0.12 to 0.30.

7. The process of claims 2-6, wherein the protease is an acid protease, in particular an acid fungal protease, such as an acid fungal protease derived from a strain of Aspergillus, in particular Aspergillus niger or Aspergillus oryzae; or a strain of Rhizomucor, in particular Rhizomucor miehei.

8. The process of claim 3-7, wherein the debranching enzyme is an isoamylase (E.C. 3.2.1.68) or pullulanase (E.C. 3.2.1.41), in particular a pullulanase derived from Bacillus sp., such as a strain of Bacillus deramificans; Bacillus acidopullulyticus or Bacillus naganoensis.

9. The process of claims 3-8, wherein the glucoamylase/pullulanase ratio determined as AGU/PUN is from 5:1 to 1:5.

10. The process of claims 1-9, wherein the fermentation is carried out using a micro-organism capable of fermenting sugars or converted sugars, such as glucose or maltose.

11. The process of claims 1-10, wherein the micro-organism is a yeast, in particular Saccharomyces cerevisae.

12. The process of claims 1-11, wherein the material to be fermented is liquefied whole grain mash or a side stream from starch processing, in particular liquefied starch with a DE of 8-10.

13. The process of claims 1-12, wherein the product is beer or wine

14. The process of claims 1-12, wherein the product is ethanol.

15. A process for the production of ethanol, comprising the steps of:
(a) milling whole grains;
(b) liquefying the product of step (a), in the presence of an alpha-amylase;
(c) saccharifying the liquefied material obtained in step (b);
(d) fermenting the saccharified material obtained in step (c) using a micro-organism, and optionally;
(e) distilling of the fermented and saccharified material obtained in step (d) providing two fraction: 1) an alcohol fraction and 2) a Whole Stillage fraction;
(f) separating the Whole Stillage into two fractions: 1) Wet Grain fraction, and 2) Thin Stillage;
(g) optionally the Thin Stillage is evaporated to provide two fractions: 1) Condensate and 2) Syrup;
wherein the steps (c) and/or (d) are carried out in the presence of carbohydrate-source generating enzyme activity and an alpha-amylase activity and the and steps (c) and (d) may be carried out either simultaneously or separately/sequentially.

16. The process of claim 15, wherein the liquefaction step comprises the following sub-steps;
b1) the hot slurry is heated to between 60-95° C., preferably 80-85° C., and at least one alpha-amylase is added;
b2) the slurry is jet-cooked at a temperature between 95-140° C., preferably 105-125° C. to complete gelatinization of the slurry;
b3) the slurry is cooled to 60-95° C. and more alpha-amylase is added to finalize hydrolysis.

17. The process of claims 15 or 16, wherein the liquefaction process is carried out at pH 4.5-6.5, in particular at a pH between 5 and 6.

18. The process of claims 15-17, wherein further a protease activity, and/or debranching enzyme activity is added during pre-saccharification or saccharification or SSF.

19. The process of claims 15-18, wherein further a protease activity and/or debranching enzyme activity is added during fermentation.

20. The process of claims 15-19, wherein steps (c) and (d) are carried out either simultaneously or separately/sequentially.

21. The process of claims 15-20, wherein an optionally ethanol recovery step is carried out after step (e).

22. The process of claim 15-21, wherein the milling step (a) is a dry milling step or wet milling step.

23. The process of claims 15-22, wherein the alpha-amylase(s) used for liquefaction in step (b) is derived from a strain of the genus Bacillus or a strain of Aspergillus.

24. The process of claims 15-23, wherein the fermentation is carried out using a micro-organism capable of fermenting sugars to ethanol.

25. The process of claim 24, wherein the micro-organism is a yeast, such as a yeast belonging to Saccharomyces spp., in particular Saccharomyces cerevisae.

26. The process of any of claims 1-25 wherein further one or more yeast cell wall degrading enzyme(s) is(are) present.

27. A composition comprising all of the following enzyme activities: carbohydrate-source generating enzyme activity, alpha-amylase activity, protease activity and debranching enzyme activity.

28. Use of a composition of claim 27 for saccharification and/or fermentation.

29. Use of a composition of claim 27 for ethanol production or for beer or wine production.

30. The composition of claim 27 wherein further one or more yeast cell wall degrading enzyme(s) is(are) present.

31. Use of a composition of claim 30 for saccharification and/or fermentation.

32. Use of a composition of claim 30 for beer or wine production

33. Use of a composition of claim 30 for ethanol production.

34. A process of producing a fermentation product, which process comprises a fermentation step, wherein the fermentation step is carried out in the presence of one or more yeast cell wall degrading enzyme(s).

35. The process of claim 34, wherein the yeast cell wall degrading enzyme(s) is(are) selected from the group including beta-1,3-glucanase, 1,3-beta-glucanase, laminarinase, xylanase, chitinase, mannanase, alpha-1,3-glucanase (mutanase), and protease.

36. The process of claim 34-35, wherein the yeast cell wall degrading enzyme is a preparation, such as the product GLUCANEX™ derived from Trichoderma harzianum.

37. The process of claims 34-36, wherein the fermentation is carried out using a micro-organism capable of fermenting sugars or converted sugars, such as glucose or maltose.

38. The process of claim 37, wherein the micro-organism is a yeast, in particular Saccharomyces cerevisae.

39. The process of claims 34-38, wherein the material to be fermented is liquefied whole grain mash or a side stream from starch processing, in particular liquefied starch with a DE of 8-10.

40. The process of claims 34-39, wherein the fermentation is carried out in the presence of further a carbohydrate-source generating enzyme, such as glucoamylase, beta-amylase or maltogenic amylase, and/or further a protease.

41. The process of claims 34-40, wherein the protease is selected from the group of fungal proteases, such as an acid fungal protease derived from a strain of Aspergillus, in particular A. niger.

42. The process of claims 34-41, wherein the protease is a bacterial protease, such as an acid, neutral or alkaline protease, such as a protease derived from a strain of Bacillus, in particular ALCALASE® or NEUTRASE®.

43. The process of claims 34-42, wherein the product is ethanol or the product is beer or wine.

44. A process of producing ethanol, wherein the process sequentially comprises the following steps
a) milling whole grains;
b) liquefying the product of step (a), in the presence of an alpha-amylase;
c) saccharifying the liquefied material obtained in step (b);
d) fermenting the saccharified material obtained in step (c) using a micro-organism, and optionally;
e) distilling the fermented and saccharified material obtained in step (d), providing two fraction: 1) an alcohol fraction and 2) a Whole Stillage fraction;
f) separating the Whole Stillage into two fractions: 1) Wet Grain fraction, and 2) Thin Stillage;
g) optionally the Thin Stillage is evaporated to provide two fractions: 1) Condensate and 2) Syrup;
wherein step (c ) and/or (d) is carried out in the presence of one or more yeast cell wall degrading enzyme(s), in particular one more enzyme activities as defined in claims 33 or 34.

45. The process of claim 44, wherein the treated Thin Stillage and/or condensate thereof is recycled to the liquefaction step b), in particular to the milled whole grain slurry.

46. The process of claims 44 or 45, wherein the liquefaction step comprises the following sub-steps:
b1) the hot slurry is heated to between 60-95° C., preferably 80-85° C., and at least one alpha-amylase is added;
b2) the slurry is jet-cooked at a temperature between 95-140° C., preferably 105-125° C. to complete gelanitization of the slurry;
b3) the slurry is cooled to 60-95° C. and more alpha-amylase is added to finalize hydrolysis.

47. The process of claims 44-46, wherein the liquefaction process is carried out at pH 4.5-6.5, in particular at a pH between 5 and 6.

48. The process of claims 44-47, wherein the cell wall degrading enzyme is added during pre-saccharification or saccharification.

49. The process of claims 44-48, wherein the yeast cell wall degrading enzyme is added during fermentation.

50. The process of claims 44-49, wherein steps (c) and (d) are carried out either simultaneously or separately/sequentially.

51. The process of claims 44-50, wherein an optionally ethanol recovery step is carried out after step (e).

52. The process of claims 44-51 wherein the milling step (a) is a dry milling step or wet milling step.

53. The process of claims 44-52, wherein the alpha-amylase used for liquefaction in step (b) is derived from a strain of the genus Bacillus or a strain of Aspergillus.

54. The process of claims 44-53, wherein the fermentation is carried out using a micro-organism capable of fermenting sugars to ethanol.

55. The process of claim 54, wherein the micro-organism is a yeast, such as derived from Saccharomyces spp., in particular Saccharomyces cerevisae.

56. The process of claims 54-55, wherein the fermentation is carried out in the presence of at least one carbohydrate-generating enzyme, in particular a glucoamylase, and further a protease.

57. The process of claim 56, wherein the protease is selected from the group of fungal proteases, such as an acid fungal protease derived from a strain of Aspergillus, in particular A. niger.

58. The process of claim 56, wherein the protease is a neutral or alkaline protease, such as a protease derived from a strain of Bacillus, in particular Alcalase® or Neutrase®.

59. The process of any of claims 34-58 wherein further at least one carbohydrate-source generating enzyme activity and at least one alpha-amylase activity are present.

60. The process of any of claims 1-26 and 34-59 where one or more process step(s) is(are) performed batch wise and/or one or more process step(s) is(are performed as a continuous flow

61. Use of a yeast cell wall degrading enzyme for saccharification and/or fermentation

62. Use of a yeast cell wall degrading enzyme for ethanol production.

63. A composition comprising a yeast cell wall degrading enzyme and at least one carbohydrate-generating enzyme, in particular a glucoamylase.

64. A composition of claim 63, said composition further comprising a protease, in particular an acid protease, especially an acid fungal protease.

65. The composition of claim 63 wherein further at least one alpha-amylase activity is present.

66. Use of a composition of claim 63 for saccharification and/or fermentation.

67. Use of a composition of claim 63 for ethanol production.

68. Use of a composition of claim 63 for beer or wine production.
